(19) United States
(12) Patent Application Publication
(10) Pub. No.: US 2008/0299073 A1
(43) Pub. Date: Dec. 4, 2008

(54) Method For Treatment Or Prophylaxis Of An Infection Using Either An Antibody Which Binds To IL-9 Or An Agent Which Stimulates Production Of Autoantibodies To Interleukin-9

(76) Inventors: Jacques Van Snick, Brussels (DE); Frank Brombacher, Cape Town (ZA); Berenice Arendse, Cape Town (ZA)

Correspondence Address: FULBRIGHT & JAWORSKI, LLP, 666 FIFTH AVE, NEW YORK, NY, 10103-3198, US

(21) Appl. No.: 11/661,737

(22) PCT Filed: Jul. 27, 2005
(86) PCT No.: PCT/US2005/026488

§ 371 Date: Jul. 8, 2008

Related U.S. Application Data
(60) Provisional application No. 60/591,798, filed on Jul. 28, 2004.
(60) Provisional application No. 60/617,177, filed on Oct. 8, 2004.

(51) Int. Cl. A61K 38/20; A61K 39/395; A61P 33/00

(52) U.S. Cl. 424/85.2; 424/158.1; 424/145.1; 424/133.1

(57) ABSTRACT

The invention relates to methods for the treatment and/or prophylaxis of infections that produce an imbalance of Th1 and Th2 CD4 cells such as Leishmaniasis, via the administration of an antibody to IL-9 or an agent that stimulates production of anti-IL-9 antibodies sufficient to neutralize native IL-9 in a subject.

### RELATED APPLICATIONS

This application claims priority of Ser. No. 60/591,798, filed Jul. 28, 2004, and Ser. No. 60/617,177, filed Oct. 8, 2004 and incorporated by references.

### FIELD OF THE INVENTION

This invention relates to methods for the treatment and/or prophylaxis of infections, such as parasitic infections, including Leishinaniasis. In particular, it relates to such methods where the infection results in the distortion of proper Th1/Th2 balance in a subject, and administration of an agent, such as antibody which binds to IL-9 or conjugates of IL-9 and a carrier protein, such as ovalbumin act to promote a balance in Th1/Th2 response.

### BACKGROUND AND PRIOR ART

Cytokines are involved in many biological functions and are major mediators of immune responses. They are associated, inter alia, with the progression and resolution of various infectious diseases. For example, tumor necrosis factor α (TNFα), interferon γ (IFNγ) and interleukin-1 (IL-1) have been associated with efficacious treatment of some parasitic diseases such as Leishmaniasis while IL-4, IL-5, IL-9 and IL-13 have been associated with the resolution of infection by T. muris.

Interleukin-9 (“IL-9” hereafter) is a glycoprotein that has been isolated from both murine and human cells. See, e.g., U.S. Pat. No. 5,208,218, incorporated by reference. This reference also teaches isolated nucleic acid molecules encoding the protein portion of the molecule, and how to express it.

Because of its restricted production by Th2 clones in vitro (Gessner, et al., Immunobiology, 189:419-435 (1993)) and its expression as part of Th2-type responses in vivo (Grencis, et al., Immunology, 74:329-332 (1991); Svetic, et al., J. Immunol., 150:3434-3441 (1993); Faulkner, et al., Infect. Immun., 66:3832-3840 (1998)), IL-9 is considered to be a Th2 cytokine that can be induced via IL-4 dependent (Kopf, et al., Nature, 362:245-248 (1993)) and IL-4 independent (Monteyne, et al., J. Immunol., 159:2616-2623 (1997)) pathways. IL-10 dependence on IL-9 production has also been described in different models. See Monteyne, et al., supra; Houssiau, et al., J. Immunol., 154:2624-2630 (1995).

Since its discovery as a T- and mast cell-growth factor produced by Th2 cells, the physiological processes in which IL-9 is known to have a role have been gradually expanded (See, for example Uyttenhove, et al., Proc. Natl. Acad. Sci. USA, 85:6934-6938 (1988); Hültner, et al., Eur. J. Immunol., 20:1413-1416 (1990); and Gessner, et al., Immunobiol., 189:419-435 (1993); Renauld, J.-C., & Van Snick, J. (1998), The Cytokine Handbook 313-331). Prominent features, disclosed by analysis of transgenic mice overexpressing IL-9, include increased susceptibility to lymphoma-genesis (Renauld, et al., Oncogene, 9:1327-1332 (1994)), intestinal mastocytosis (Godfraind, et al., J. Immunol, 160:3989-3996 (1998)), expansion of the B-1 lymphocyte population (Godfraind, et al., J. Immunol., 160:3989-3996 (1998)), bronchial hyper-responsiveness (Temann, et al., J. Exp. Med., 188:1307-1320 (1998), and McLane, et al., Am J. Resp. Cell. Mol., 19:713-720 (1998)) and airway eosinophilia (Dong, et al., Eur. J. Immunol., 29:2130-2139 (1999)). In line with these observations, genetic analyses have revealed a linkage between both IL-9 and IL-9 receptor (IL-9R) genes and human asthma (Holroyd, et al., Genomics, 52:233-235 (1998); Marsh, et al., Science, 264:1152-1156 (1994)), a finding that was confirmed with respect to IL-9 in murine models (Nicolaides, et al., Proc. Natl. Acad. Sci. USA, 94:13175-13180 (1997)).

Various uses of IL-9 are disclosed in, e.g., U.S. Pat. Nos. 5,164,317 (proliferation of mast cells); 5,246,701 and 5,132,109 (enhancing production of IgG and inhibiting production of IgE), in addition to its first recognized utility, which is as a T cell growth factor. Exemplary of the vast scientific literature on IL-9 are Van Snick, et al, J Exp Med. 169(1):363-368 (1989) (cDNA for the murine molecule, then referred to as P40). Houssiau, et al., J. Immunol., 148(10):3147-3151 (1992) (IL-2 dependence of IL-9 expression in T lymphocytes). Renauld, et al., Oncogene, 9(5):1327-1332 (1994) (effect on thymic lymphomas); Renauld, et al, Blood, 85(5):1300-1305 (1995) (anti-apoptotic factor for thymic lymphoma); U.S. Pat. No. 5,830,454 (treatments of cell mediated autoimmune disorders); and U.S. Pat. No. 5,935,929 (treating or preventing interstitial lung disease). Review articles may be found at, e.g., Renauld, et al., Cancer Invest., 11(5):635-640 (1993); Renauld, et al., Adv. Immunol., 54:79-97 (1993); Demoulin, et al., Int. Rev. Immunol., 16:345-364 (1998).

While detrimental in asthma, elevated production of Th2 cytokines has been reported to correlate with resistance to certain parasite infections (Finkelman, et al., Annu. Rev. Immunol., 15:505-533 (1997)). IL-9, for example, was found to enhance murine resistance to infection with the caecal dwelling nematode T. muris (Faulkner, et al., Infect. Immun., 66:3832-3840 (1998)). This resistance was associated with high IgE and IgG1 levels, as well as with pronounced intestinal mastocytosis.

The absence of T cell help has previously been suggested to be crucial for B cell tolerance toward self-proteins (Dalum, et al., J. Immunol., 157:4786-4804 (1996)). Using bovine leuteinizing hormone (LH) as a self protein coupled to ovalbumin (OVA), Johnson, et al. (J. Anim. Sci., 66:719-726 (1988)) were able to induce high titers of autoantibodies against LH, causing cows to become anestrous. Similarly, a vaccine that prevents pregnancy in women was developed by coupling either human chorionic gonadotropin (hCG) or ovine luteinizing hormone to either tetanus or diphtheria toxoids (Talwar, et al., Proc. Natl. Acad. Sci. USA, 91:8532-8536 (1994)). More recently, immunization with a fusion protein of an OVA epitope and mouse TNFα was found to prevent experimental cachexia and collagen-induced arthritis in mice (Dalum, et al., Nature Biotechnology, 17:666-669 (1999)).

Earlier attempts to generate autoantibodies capable of regulating biological processes, were successfully carried out mainly with hormones (Johnson, et al., J. Anim. Sci. 66:719-726 (1988); Talwar, et al., Proc. Natl. Acad. Sci. USA, 91:8532-8536 (1994)), hormone receptors (Chackerian, et al. Proc. Natl. Acad. Sci. USA, 96:2773-2778 (1999)) or cellular components (Dong, et al., J. Exp. Med., 179:1243-1252 (1994) and Dalum, et al., Mol. Immunol., 34:1113-1120 (1997)). These observations were recently extended to cytokines, with reports of anti-IFNα induction in AIDS patients (Zagury, et al., Biomed. Pharmacother, 53:90-92 (1999)) and of anti-TNFα vaccination in mice, the latter preventing cachexia and rheumatoid arthritis (Dalum, et al., Nature Biotechnology, 17:666-669 (1999)).

In U.S. Pat. No. 6,645,486, incorporated herein by reference in its entirety, methods for generating antibodies specific for interleukins are disclosed. Specifically, it is shown therein that administration of conjugates of IL-9 and other molecules, such as IL-9/ovalbumin conjugates, generates anti-IL-9 antibodies, in vivo. The antibodies generated have a neutralizing effect on IL-9 and are useful in treating pathological conditions where regulation of IL-9 is warranted, such as eosinophilia and allograft rejection.

It has also been shown (Khan, et al, Infect. Immun., 71:2430-2438 (2003)); incorporated by reference, that treatment with exogenous antibody to IL-9 can have an effect similar to the production of autoantibodies to IL-9.

Disregulated expression of IL-9 induces profound perturbations in multiple hematopoietic cell lineages, resulting in diverse phenotypes. Thus, ubiquitous expression of an IL-9 transgene is associated with lymphomagenesis, enhanced immunoglobulin expression (Renauld, et al, Oncogene, supra), expansion of a B1 cell lymphocyte subset (Vink, et al., J. Exp. Med., 189:1413-1423 (1999)), mastocytosis, and eosinophil maturation (Louahed, et al., Blood, 97:1035-1042 (2001)), and parasitic worm expulsion (Faulkner, et al., supra; Faulkner, et al., Eur. J. Immunol., 27:2536-2540 (1997)). Furthermore, expression of IL-9 transgenes under the control of a lung-specific promoter leads to severe airway inflammation with infiltration of eosinophils and lymphocytes, mast cell hyperplasia and increased sub-epithelial collagen deposition. Temann, et al., J. Clin. Invest., 109:29-39 (2002). IL-9 deficient mice also exhibit severe impairment of goblet cell hyperplasia and mastocytosis in a pulmonary granuloma model, although pulmonary fibrosis, eosinophil and lymphocyte infiltration and development of pulmonary Th2 responses is normal. Townsend, et al., Immunity, 13:573-583 (2000).

Experimental murine Leishmaniasis is a paradigmatic example of the relationship between the genetic factors that control T helper cell differentiation and the outcome of the disease. Resistant strains of mice (healers strains) like C57BL/6 develop predominantly Th1 responses with high IFN-γ, low IL-4 production and protective cellular immune responses, while genetically susceptible strains (non-healers strains) like BALB/c, develop predominantly Th2 responses with high IL-4 and low IFN-g production, which results in exacerbation of the disease. Heinzel, et al., Journal of Experimental Medicine, 169:59-72 (1989).

Leishmania-induced Th2 cytokine responses include IL-4, IL-5, IL-9 and IL-13. IL-4 is a principal disease-promoting factor in cutaneous Leishmaniasis (Louis, et al., Curr. Opin. Immunol., 10:459-464 (1998); Fowell & Locksley, Bioessays, 21:510-518 (1999)) and neutralization of IL-4 in vivo by monoclonal antibodies converts non-healers to healers. (Sadick, et al., J. Exp. Med. 171:115-127 (1990)). BALB/c mice deficient for Il-4, IL-13, IL-4Ra or STAT6 are able to control acute infection with impaired Th2 responses. See Kopf, et al., J. Exp. Med., 184:1127-1136 (1996); Matthews, et al., J. Immunol., 164:1458-1462 (2000); Mohrs, et al., J. Immunol., 162:7302-7308 (1999); and Stamm, et al., J. Immunol., 161:6180-6188 (1998), respectively. These studies suggest that both IL-4 and IL-13 contribute to the susceptible phenotype, which seems to depend on the Leishmania strain used. See Kopf, et al., supra; Noben-Trauth, et al., Science, 271:987-990 (1996); and Noben-Trauth, et al., The Journal of Immunology, 162:6132-6140 (1999).

The exact role of IL-13 in Leishmaniasis remains unclear. IL-13 has disease promoting functions during acute Leishmaniasis and possible protective functions during the later chronic disease. See Matthews, et al., supra; Mohrs, et al., supra. IL-5 also plays a minor role in the overall susceptibility in Leishmania major infected BALB/c mice. See Kopf, et al. Immunol. Rev., 148:45-69 (1995).

IL-9 is induced by L. major infection and transiently expressed during the first days after infection. From 4 weeks post-infection onwards, IL-9 synthesis is only observed in susceptible BALB/c mice, but not in resistant C57BL/6 or DBA mice (Gessner, et al., supra; Nashed, et al., Microbes. Infect., 2:1435-1443 (2000)). Expression correlates with the expansion of antigen-specific Th2 cells and purified CD4+ T cells are capable of producing IL-9 during polyclonal or antigen-specific restimulation (Gessner, et al., supra).

Although IL-9 has been shown to be expressed during L. major infection, the potential role of IL-9 in Leishmaniasis has not been explored. In the disclosure that follows, the role of the Th2 cytokine IL-9 is examined.

### BRIEF DESCRIPTION OF THE FIGURES

FIG. 1A depicts the course of L. major infection in IL-9/OVA vaccinated mice and control mice.

FIG. 1B depicts the parasite load in draining popliteal lymph nodes.

FIG. 1C depicts the parasite load in infected footpads

### DETAILED DESCRIPTION OF PREFERRED EMBODIMENTS

### Example 1

Groups of healer (C57BL/6), and non-healer (BALB/c) strains of mice were prepared. Each group contained eight animals. The animals all received three injections of IL-9 that had been cross linked to OVA, in accordance with Richard, et al., Proc. Natl. Acad. Sci. USA, 97:747-772 (2000), or U.S. Pat. No. 6,645,486, both of which are incorporated by reference. In brief, however, equimolar amounts of purified IL-9 and OVA were combined with gluteraldehyde, at a final concentration of 50 mM, in 0.1M phosphate buffer, pH 7. The reaction was carried out by shaking the mixture for 3 hours at room temperature, and then overnight, at 4° C.

The mice were then injected, subcutaneously, with 100 μl of a 1:1 mixture of the complexes in phosphate buffered saline, and complete Freund's adjuvant.

Booster injections were administered two and four weeks later. Control mice received equivalent amounts of ovalbumin, in Freund's adjuvant. Three weeks after the last immunization, the immunized and control mice were anesthetized, and injected, in the left hind footpad, with 2×10⁶ metacyclic promastigotes of Leishmania major (MHOM/IL/81/FEBNI, “LIT” hereafter), to a final volume of 50 μl in HBSS. The strain was maintained by continuous passage in BALB/c mice, as described by Mohrs, et al., J. Immunol., 162:7302-7308 (1999), incorporated by reference. Parasites were isolated from skin lesions of the infected animals.

### Example 2

Experiments were carried out to investigate if IL-9 was involved in leishmaniasis. Three weeks after the last immunization, the immunized and control mice were anesthetized, and injected, in the left hind footpad, with 2×10⁶ metacyclic promastigotes of Leishmania major (MHOM/IL/81/FEBNI, “LIT” hereafter), to a final volume of 50 μl in HBSS. The strain was maintained by continuous passage in BALB/c mice, as described by Mohrs, et al., J. Immunol., 162:7302-7308 (1999), incorporated by reference. Parasites were isolated from skin lesions of the infected animals.

Following the injection with LIT described supra, the course of infection was monitored weekly by measuring infected, and non-infected hind footpads. Values were expressed as mean ±SD of 8 mice per group, of representatives of 3 different experiments. Onset of ulceration and necrosis were noted.

Control BALB/c mice developed massive footpad swellings, with ulceration and necrosis, starting at the third week following inoculation. All mice had to be killed 8 weeks post infection, due to the extent of the disease progression.

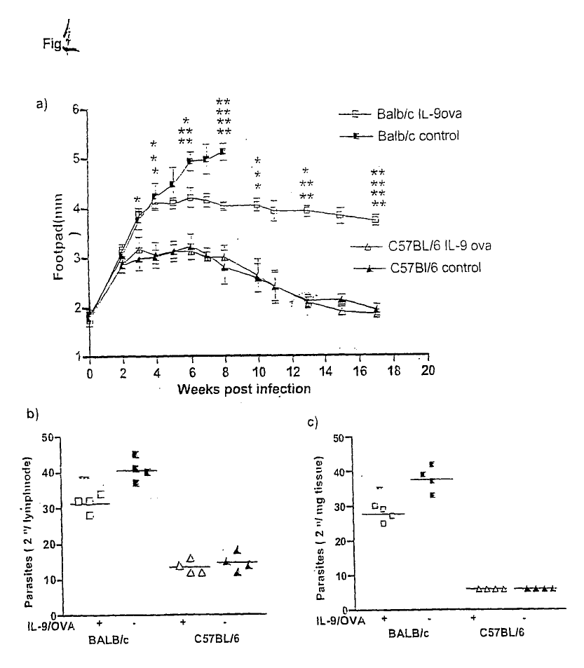

In contrast, the BALB/c mice which had received the conjugate injection (IL-9-OVA), the footpad swelling stabilized, at a moderate level, within the first 4 weeks. This can be seen in FIG. 1A.

### Example 3

Experiments were also carried out to determine parasite burden in infected mice in draining popliteal lymphnodes, and in the infected footpad. Parasite burden from homogenized organs was determined by 2-fold limiting dilutions in Schneiders medium. The results are presented in FIGS. 1B and 1C. In both situations, the parasite burden in IL-9-OVA immunized, BALB/c mice was significantly lower than in infected, BALB/c controls, at 8 weeks post infection.

In follow up independent experiments, data collected at weeks 5 and 9, these results were confirmed. The BALB/c mice which were immunized with the conjugate more than doubled their life span, with ulceration and necrosis only occuning at the tenth week and thereafter.

At the end stage of the disease, control OVA immunized BALB/c mice showed drastic pathology, including severe bone destruction in the footpad, and viscerilization in other organs, including spleen and liver.

In contrast, healer strain C57BL/6 developed transient and moderate swelling during the first week and then developed resistance to L. major infection. There was no significant different observed in control OVA-immunized mice, as compared to IL-9-OVA immunized C57BL/6 mice.

These data provide evidence for a beneficial role of anti-IL-9 vaccination during L. major infection in the non-healer BALB/c strain.

### Example 4

The L. major-induced T-helper response was investigated, to determine the possible mechanisms involved in the delayed disease progression shown by IL-9-OVA immunized mice. Specially, Th1/type1 and Th2/type2 responses were determined to see if there was a change in the T helper response.

First, CD4⁺ T cells were isolated from lymph nodes and purified via positive selection with magnetic mouse CD4 specific beads, in accordance with Muller, et al., J. Immunol., 167:3346-3353 (2001). The positive selection protocol yielded enriched CD4⁺ cells with greater than 90% purity, as determined via standard FACS.

Differentiation of the enriched CD4⁺ cells was induced in accordance with Mohrs, et al., Infect. Immunol., 68:1773-1780 (2000), incorporated by reference. In brief, 2×10⁶ cell/ml samples of CD4⁺ cells were stimulated with anti-CD3 antibodies, frozen-and-thawed L. major antigen, or medium only, and incubated at 37° C., in a 7% CO₂ atmosphere. Supernatants were collected 40 hours later, and then cytokine concentrations for IFN-γ, IL-4, IL-9, and IL-13 were determined in culture supernatants, using standard ELISA methods as described, e.g., by Dai, et al., J. Immunol., 158:5297-5304 (1997). Samples were taken from 4 mice per group, at 5 weeks, and 8 weeks post infection. Briefly, supernatants and appropriate cytokine standards were used in threefold serial dilutions and detection limits for IFN-γ, IL-9, IL-13 was 46 pg/ml and IL-4:2 pg/ml.

In agreement with reduced parasite burden, mitogen, or antigen stimulated CD4⁺ T cells from the IL-9-OVA immunized BALB/c mice produced significantly higher levels of IFN-γ at week 8, and strikingly lower IL-4 levels at weeks 5 and 8, as compared to control mice. These results were consistent with the other Th 2 effector cytokines, i.e., IL-9 and IL-13, with significantly lower production in the vaccinated BALB/c mice.

Healer strain C57BL/6, as expected, showed a predominantly L. major specific, Th1 polarized response, showing high IFN-γ and very low IL-4 production, without an antigen specific effect resulting from the vaccination. The CD4⁺ T cells from BALB/c mice produced considerable amounts of IL-9 (ng range) while C57BL/6 derived, CD4⁺ T cells showed marginal IL-9 production. These results were expected, as both IL-9 and IL-13 are Th2 derived cytokines, lacking for the most part in L. major infected, C57BL/6 mice.

### Example 5

Antigen-specific antibody responses were studied to determine the in vivo, downstream consequences of the immunizations described supra to determine the role of IL-9 L. major infections.

Sera were collected 5 and 8 weeks post infection (2 weeks after the last immunization), from immunized and control animals, of both strains. Antigen-specific Ig ELISA was performed according to Mohrs et al., J. Immunol., 162:7302-7308 (1999). Levels of L. major-specific serum antibody isotypes were measured by coating plates with freeze-and-thawed preparations of L. major promastigotes and detected by commercially available anti-mouse Ig-isotype-specific polyclonal antibodies. Serum samples and immunoglobulin standards for total IgE were used in 10-fold limiting dilutions. Total IgE was determined with monoclonal antibody 84.1 C for coating and alkaline phosphatase labeled EM95.3 for detection. The detection limit for IgE was 10 ng/ml.

The results paralleled what was seen in the T helper differentiation profile, i.e., the IL-9-OVA immunized BALB/c mice showed increased type 1 antibody responses, and impaired type 2 antibody responses, as compared to control BALB/c mice. This was demonstrated via endpoint dilution analysis, of antigen-specific IgG2a, and IgG2b, as compared to IgG1 and IgE titers.

With respect to the C57BL/6 mice, there were not significant differences in the antibody responses, depending upon immunization or not.

These results provide evidence that following the neutralization of endogenous IL-9, there is a shift toward a protective Th1/type 1 response.

### Example 6

It is well known that macrophages are the major cellular host for L. major. It is here that amastigotes propagate in the phagolysosome. Nitric oxide (NO) is the crucial killing effector molecule against leishmaniasis, produced by IFN-γ stimulated, and iNOS induced classical macrophages. In order to determine if IL-9-OVA immunization had influence on L. major specific killing effector functions, macrophages were isolated from thioglycollate elicited, peritoneal exudates from mice 8 weeks after infection, using standard methods. These were then cultured, in triplicate, at concentrations of 2×10⁶ cells/ml, in 96 well plates, for 4 hours. Any plastic adherent macrophages were stimulated with LPS, at 10 ng/ml, or 100 U/ml of IFN-γ for 48 hours. At this point, the nitric oxide concentration in the supernatants was measured by the Griess reaction, in accordance with Holscher, et al., Infect. Immun., 66:1208-1215 (1998). Urea levels were also measured (through the determination of arginase levels) in accordance with Corroliza, et al., J. Immunol. Methods, 174:231-235 (1994). Both of these papers are incorporated by reference.

The macrophages from IL-9-OVA immunized BALB/c mice showed small, but significant increased nitric acid synthase induced NO production, as compared to controls. This was verified by showing a stark reduction in urea production in the immunized animals. Urea is a recognized side product of arginase I activity.

The differential outcome is explained by the fact that there is competition between iNOS and arginase I for the common substrate, L-arginine.

There was no effect on NO production in C57BL/6 mice

These data suggest that the increase in NO observed as a result of IL-9 immunization has a direct effect on the killing of amastigotes in macrophages of L. major infected mice.

### Example 7

These experiments were carried out to determine if anti IL-9 vaccination is dependent on IL-4 or IL-13 mediated functions. To do this, L. major infection studies were performed in BALB/c IL-9-OVA immunized, IL-4Ra deficient mice, using the immunization protocol of example 1. These mice are unresponsive to IL-4 and IL-13, because the IL-4Ra chain is a crucial component of the receptors for IL-4 and IL-13. See Brombacher, Bioassays, 22:646-656 (2000).

The vaccination with IL-9-OVA conjugates was as effective in the IL-4Ra deficient mice as the normal BALB/c mice, with half maximal inhibition at 2.5 units/ml of IL-9, obtained at a mean serum dilution of 5×10⁴.

In the absence of IL-4Ra, these BALB/c mice were resistant, showing only slightly increased footpad swelling as compared to C57BL/6 mice.

While immunization with the conjugate did have a protective effect in BALB/c mice, which was observed by delayed footpad swelling, disease progression, and reduced parasite burden, all as described supra, no effect was seen in the IL-9 vaccinated, BALB/c IL-4Ra deficient mice, as compared to controls.

All of the CD4⁺ T cells' IFN-γ, IL-4 and IL-9 responses were assayed, as described supra, after either antigen or CD3-specific restimulation. The responses were similar between IL-9, and sham immunized IL-4Ra deficient mice. No IL-9 could be found.

Taken as a whole, these data suggest that the BALB/c IL-4Ra deficient strain naturally produces a TH-1 type response to L. major and therefore IL-9 vaccination has no influence on the balance of T helper differentiation. EXAMPLE 8

In follow up experiments, conjugates of IL-9 and ovalbumin, as well as conjugates of IL-9 and transferrin were prepared.

The conjugates of ovalbumin and IL-9 were made by combining 1 mg of IL-9 and 2 mg of ovalbumin, overnight, in the presence of 20 mM gluteraldehyde in 0.15M phosphate buffer, at pH 7.

To make the IL-9/transferrin conjugates, 200 μg of IL-9, and 600 μg of human transferrin were combined, again in the presence of 20 mM gluteraldehyde, in phosphate buffer. The reaction was allowed to proceed overnight. Formed complexes were dialyzed against PBS, and were then used in animal studies.

In the animal studies, C57BL/6 mice received subcutaneous injections of 2 ggs of the conjugates, three times, at two week intervals. The conjugates were combined with Complete Freund's adjuvant in the first immunization, and Incomplete Freund's adjuvant in the final two. Serum samples were taken from the mice, two weeks after the final injection.

Serum samples were then tested, on TS1 cells, to determine whether IL-9 neutralizing antibodies were present. Uyttenhove, et al., Proc. Natl. Acad. Sci. USA, 85:6934-6938 (1988), incorporated by reference, have shown that Active IL-9 is required to sustain TS1 cell growth.

The TS1 cells used had been grown in DMEM medium supplemented with 10% fetal calf serum, 1.5 mM L-glutamine, 0.24 mM L-asparagine, 0.55 mM L-arginine, 50 μM 2-mercaptoethanol, and 200 U/ml of murine IL-9 (5 ng/ml). The cultures were diluted, 100-1000 fold, twice per week.

For the activity assays, sera were diluted with TS1 cell culture medium lacking IL-9, at a 1/40 ratio. Controls were diluted 1/10.

Equal amounts of the diluted serum samples (50 μl) were then incubated at room temperature for 1 hour with 50 μl of IL-9 (200 pg/ml), prior to adding the cells.

TS1 cells were washed, twice, with the culture medium lacking IL-9 and a 100 μl sample of 3000 TS1 cells were added to the IL-9 containing serum samples, and the mixtures were incubated for 3 days, before hexoaminidase activity was measured, in accordance with Uyttenhove, et al., supra.

The results, which follow, show that IL-9 conjugates, other than IL-9 ovalbumin conjugates, may be used to prepare IL-9 neutralizing antibodies:

| | TS1 cell growth (Abs. 405 nm) |
|---|---|
| No IL-9 | 0.157 |
| IL-9 | 1.285 |
| IL-9 + serum from IL-9-OVA | 0.192 |
| mouse diluted 1/40 | |
| IL-9 + serum from IL-9-h | 0.696 |
| Transferrin mouse diluted 1/40 | |

### Example 9

Two groups of healer (C57BL/6), and two groups of non-healer (BALB/c) strains of mice are prepared with one group of each acting as a non-treated control group. Each group contains eight animals. Both treated and control mice are anesthetized, and injected, in the left hind footpad, with 2×10⁶ metacyclic promastigotes of Leishmania major (MHOM/IL/81/FEBNI, “LIT” hereafter), to a final volume of 50 μl in HBSS.

One week after infection, a course of treatment is started in one group of healer and one group of non-healer mice. The course of treatment consists of an initial p.i. injection of 1 mg. of anti-IL-9 monoclonal antibody (MM9A1 or MM9C1) on day zero followed by injections of 0.5 mg. of antibody every other day until day 20.

Isotype-matched control antibodies, B8401H5 (a IgG1 anti-TNP) may serve as a control for MM9A1 and C1405F9 a IgG2a anti-TNP as a control for MM9C1.

Following the injection with LIT described supra, the course of infection is monitored weekly by measuring infected, and non-infected hind footpads. Values are expressed as mean ±SD of 8 mice per group, of representatives of 3 different experiments. Onset of ulceration and necrosis are noted.

Control BALB/c mice should develop footpad swellings, with ulceration and necrosis, starting at the third week following inoculation. In contrast, healer strain C57BL/6 should develop transient and moderate swelling during the first week and then develop resistance to L. major infection.

Antibody treatment should have no effect in the infected healer strain mice however the treatment of the non-healer BALB/c mice should result in a stabilized footpad swelling, at a moderate level, within the first 4 weeks.

These data will provide evidence for a beneficial role of the use of anti-IL-9 antibody for the treatment of L. major infection in a susceptible individual.

The foregoing examples set forth various features of the invention, which relate, inter alia to a method of treatment or prophylaxis of a parasitic infection, wherein said parasitic infection induces production of IL-9, by immunizing a subject with an agent that stimulates production of anti-IL-9 antibodies or by treating the subject with antibodies to IL-9. It is preferred that the subject is a mammal, more preferably that the subject is a human being. Preferably, the agent administered is a conjugate of IL-9 and ovalbumin or transferrin and the antibody to IL-9 is monoclonal. If the subject if a human it is most preferable that the monoclonal antibody is humanized. The IL-9 can be either from the same species as said subject or from a different species.

Those of skill in the art appreciate that many methods are suitable for conjugating a carrier and a conjugation partner. The partners in a conjugate may be treated with a cross-linking agent, e.g., glutaraldehyde, carbodiimide or bis-diazobenzidine. The conjugation partners may also be modified to permit or enhance the formation of conjugates. For example, the carrier may be substituted with maleimide, e.g., a maleimide-substituted OVA, so that it is suitable for forming a conjugate with an interleukin having free SH groups, e.g., a iminothyolane-treated IL-9. See U.S. Pat. No. 6,645,486, referred to supra, for examples.

The conjugate or antibody can be administered alone, or in combination with a therapeutically effective amount of an anti-parasite drug. The amount of agent will vary, but a dose from about 10 μg to 100 μg per dose, more preferably about 50-100 μg is contemplated. The subject may receive the medicament at weekly, monthly, or longer intervals.

The preferred method of administration is subcutaneous injection, but the agent may also be administered by any of the standard means including, but not being limited to, intramuscular, intravenous, oral, intradermal and other modes known to the art. Those of skill in the art are aware of many methods that are useful for immunizing subjects which need not be set forth herein. In the invention described herein, the subjects are preferably immunized with an amount of an antigen that stimulates production of anti-IL-9 antibodies or an amount of the anti-IL-9 antibody to neutralize the IL-9 produced by the infection. The antigen or antibody may be incorporated into any conventional pharmaceutically acceptable vehicle or diluent (see, e.g., Remington's Pharmaceutical Sciences (19th Ed) (Genarro, ed. (1995) Mack Publishing Co., Easton, Pa.)). For example, the agent may be administered with, e.g., buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with, e.g., alum or alum containing formulations are exemplary of suitable diluents. The IL-9 conjugate may also be administered with an adjuvant. Preferred adjuvants are those used routinely in the art, e.g., Freund's Incomplete Adjuvant or Freund's Complete Adjuvant and Merck Adjuvant 65. Immunization with the agent may also be combined with the administration of other components involved with the recruitment of mast cells, eosinophils or TH2 cells, e.g., MCP-1, MCP-3, MCP-4, Eotaxin, MDC/TARC and I-309, see U.S. Pat. No. 5,824,551.

The invention is also directed to promoting a protective Th1/type 1 immune response in a subject either suffering from or susceptible to an infection that produces a non-protective Th2/type 2 immune response by administering an agent that either stimulates production of anti-IL-9 antibodies or by the direct treatment with anti-IL-9 antibodies. Infections that are know to cause a non-protective or aggravating Th2/type 2 immune response are respiratory syncytial virus (RSV), human immunodeficiency virus (HIV), rhinovirus, hepatitis C virus, Helicobacter pylori, Mycobacterium ulcerans (Buruli ulcer), Mycobacterium paratuberculosis in cattle (Johne's disease). In addition, improved macrophage effector function and the abrogation of iNOS function are also features of the invention.

The invention is also directed towards a method for regulating TH1/Th2 balance in a subject suffering from an imbalance of Th1 and Th2 CD4+ cells as a result of an infection, comprising administering an agent that stimulates production of anti-IL-9 antibodies or by the direct treatment with anti-IL-9 antibodies, which results in neutralizing native IL-9 in said subject. Preferably, the agent is a conjugate of IL-9 and transferrin or ovalbumin or a monoclonal antibody that neutralizes IL-9.

Other features of the invention will be known to the skilled artisan, and need not be reiterated here.

The terms and expression which have been employed are used as terms of description and not of limitation, and there is no intention in the use of such terms and expression of excluding any equivalents of the features shown and described or portions thereof, it being recognized that various modifications are possible within the scope of the invention.

We claim:

1. A method for treatment or prophylaxis of an infection, wherein the causative agent of said infection induces production of interleukin-9, said method comprising administering to a subject in need thereof an amount of an antibody to IL-9 or an agent which stimulates production of anti-IL-9 antibodies in said subject.

2. The method of claim 1, wherein said infection is a parasitic infection.

3. The method of claim 2, wherein said parasitic infection is Leishmaniasis.

4. The method of claim 1, wherein said agent is a conjugate of IL-9 and a carrier protein.

5. The method of claim 4, wherein said carrier protein is transferring or albumin.

6. The method of claim 1, wherein said agent is an antibody which binds to IL-9.

7. The method of claim 6, wherein said antibody is a monoclonal antibody.

8. The method of claim 7, wherein said monoclonal antibody is humanized.

9. The method of claim 1, wherein said subject is a human being.

10. The method of claim 1, wherein said agent is administered by intravenous subcutaneous, intramuscular injection, orally, or intradermally.

11. The method of claim 1, wherein said agent is administered in an amount ranging from about 10 μg to 100 μg per dose.

12. The method of claim 4, wherein said IL-9 in said conjugate is from a species different from said subject.

13. The method of claim 4, wherein said IL-9 in said conjugate is from the same species as is said subject.

14. The method of claim 1, further comprising promoting a protective Th1/type 1 immune response in said subject, improving macrophage effector function, or increasing or stimulating iNOS function.

15. The method of claim 2, further comprising administering to said subject a therapeutically effective amount of an anti-parasite drug.

16. The method of claim 15, wherein said anti-parasite drug is a drug effective against Leishmania.

17. A kit useful in treatment or prophylaxis of a parasitic infection, comprising a separate portion of each of (i) an agent which stimulates production of anti IL-9 antibodies, or an antibody which binds to IL-9, (ii) an anti-parasite drug, and a container means for holding (i) and (ii).

18. The kit of claim 17, wherein (ii) is a drug effective against Leishmania.

19. The kit of claim 17, wherein (i) is a conjugate of IL-9 and transferrin or ovalbumin.

20. A method for regulating Th1/Th2 balance in a subject in need thereof, comprising administering to a subject suffering from an imbalance of Th1 and Th2 CD4 cells, an amount of an antibody to IL-9 or an agent which stimulates production of antibodies which neutralize native interleukin-9 in said subject, in an amount sufficient to alleviate said Th1/Th2 imbalance.

21. The method of claim 20, wherein said agent is a conjugate of interleukin-9 and a carrier protein.

22. The method of claim 21, wherein said carrier protein is transferring or albumin.
